(19) United States
(12) Patent Application Publication
(10) Pub. No.: US 2011/0016539 A1
(43) Pub. Date: Jan. 20, 2011

(54) GENOME EDITING OF NEUROTRANSMISSION-RELATED GENES IN ANIMALS

(75) Inventors: Edward Weinstein, St. Louis, MO (US); Xiaoxia Cui, St. Louis, MO (US); Phil Simmons, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,620

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. A01K 67/027; A01K 67/00; C12N 5/07; G01N 33/00

(52) U.S. Cl. 800/3; 800/13; 800/15; 800/14; 800/17; 435/325; 435/350; 435/351; 435/366; 435/363; 435/352; 435/353

(57) ABSTRACT

The present invention provides genetically modified animals and cells comprising edited chromosomal sequences encoding proteins that are associated with neurotransmission disorders. In particular, the animals or cells are generated using a zinc finger nuclease-mediated editing process. Also provided are methods of using the genetically modified animals or cells disclosed herein to screen agents for toxicity and other effects.

### CROSS-REFERENCE TO RELATED APPLICATIONS

This application claims the priority of U.S. provisional application number 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, US provisional application number 61/263,904, filed November 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified animals or cells comprising at least one edited chromosomal sequence encoding a neurotransmission-related protein. In particular, the invention relates to the use of a zinc finger nuclease-mediated process to edit chromosomal sequences encoding neurotransmission-related proteins in animals or cells.

### BACKGROUND OF THE INVENTION

Several genes have been associated with complex disorders of neurotransmission, based on a growing body of research. However, the progress of ongoing research into the causes and treatments of these neurotransmission disorders is hampered by the onerous task of developing an animal model which incorporates the genes proposed to be involved in the development or severity of the disorders.

Conventional methods such as gene knockout technology may be used to edit a particular gene in a potential model organism in order to develop an animal model of a neurotransmission disorder. However, gene knockout technology may require months or years to construct and validate the proper knockout models. In addition, genetic editing via gene knockout technology has been reliably developed in only a limited number of organisms such as mice. Even in a best case scenario, mice typically show low intelligence, making mice a poor choice of organism in which to study complex disorders of neurotransmission and behavior. Ideally, the selection of organism in which to model a complex neurotransmission disorder should be based on the organism's ability to exhibit the characteristics of the disorder as well as its amenability to existing research methods.

The rat is emerging as a genetically malleable, preferred model organism for the study of neurotransmission disorders, particularly because these disorders are not well-modeled in mice. Rats are a superior choice compared to mice as model organisms for the study of human diseases of neurotransmission such as psychiatric disease due to their higher intelligence, complex behavioral repertoire, and observable responses to behavior-modulating drugs, all of which better approximate the human condition. Further, the larger physical size of rats relative to mice facilitates experimentation that requires dissection, in vivo imaging, or isolation of specific cells or organ structures for cellular or molecular studies of these neurotransmission diseases.

A need exists for animals with modification to one or more genes associated with human neurotransmission disorders to be used as model organisms in which to study these disorders. The genetic modifications may include gene knockouts, expression, modified expression, or over-expression of alleles that either cause or are associated with neurotransmission diseases in humans. Further, a need exists for modification of one or more genes associated with human neurotransmission disorders in a variety of organisms in order to develop appropriate animal models of neurotransmission disorders such as amylotropic lateral sclerosis, spinocerebellar ataxias, including SCA2, Alzheimer's, autism, mental retardation, Rett's syndrome, fragile X syndrome, depression, schizophrenia, and bi-polar disorders.

### SUMMARY OF THE INVENTION

One aspect of the present disclosure encompasses a genetically modified animal comprising at least one edited chromosomal sequence encoding a neurotransmission-related protein.

A further aspect provides a non-human embryo comprising at least one RNA molecule encoding a zinc finger nuclease that recognizes a chromosomal sequence encoding a neurotransmission-related protein, and, optionally, at least one donor polynucleotide comprising a sequence encoding a neurotransmission-related protein.

Another aspect provides an isolated cell comprising at least one edited chromosomal sequence encoding a neurotransmission-related protein.

Yet another aspect encompasses a method for assessing the effect of an agent in an animal. The method comprises contacting a genetically modified animal comprising at least one edited chromosomal sequence encoding a neurotransmission-related protein with the agent, and comparing results of a selected parameter to results obtained from contacting a wild-type animal with the same agent. The selected parameter is chosen from (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); and (g) efficacy of the agent or its metabolite(s).

Still yet another aspect encompasses a method for assessing the therapeutic potential of an agent in an animal. The method includes contacting a genetically modified animal comprising at least one edited chromosomal sequence encoding a neurotransmission-related protein with the agent, and comparing the results of a selected parameter to results obtained from a wild-type animal with no contact with the same agent. The selected parameter may be chose from a) spontaneous behaviors; b) performance during behavioral testing; c) physiological anomalies; d) abnormalities in tissues or cells; e) biochemical function; and f) molecular structures.

Other aspects and features of the disclosure are described more thoroughly below.

### DETAILED DESCRIPTION OF THE INVENTION

The present disclosure provides a genetically modified animal or animal cell comprising at least one edited chromosomal sequence encoding a protein associated with neurotransmission. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the protein associated with neurotransmission generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with neurotransmission using targeted zinc finger nuclease technology is rapid, precise, and highly efficient.

### (I) Genetically Modified Animals

One aspect of the present disclosure provides a genetically modified animal in which at least one chromosomal sequence encoding a neurotransmission-related protein has been edited. For example, the edited chromosomal sequence may be inactivated such that the sequence is not transcribed and/or a functional neurotransmission-related protein is not produced. Alternatively, the edited chromosomal sequence may be modified such that it codes for an altered neurotransmission-related protein. For example, the chromosomal sequence may be modified such that at least one nucleotide is changed and the expressed neurotransmission-related protein comprises at least one changed amino acid residue (missense mutation). The chromosomal sequence may be modified to comprise more than one missense mutation such that more than one amino acid is changed. Additionally, the chromosomal sequence may be modified to have a three nucleotide deletion or insertion such that the expressed neurotransmission-related protein comprises a single amino acid deletion or insertion, provided such a protein is functional. The modified protein may have altered substrate specificity, altered enzyme activity, altered kinetic rates, and so forth. Furthermore, the edited chromosomal sequence may comprise an integrated sequence and/or a sequence encoding an orthologous protein associated with a neurotransmission-related disorder. The genetically modified animal disclosed herein may be heterozygous for the edited chromosomal sequence encoding a protein associated with a neurotransmission-related disorder. Alternatively, the genetically modified animal may be homozygous for the edited chromosomal sequence encoding a protein associated with a neurotransmission-related disorder.

In one embodiment, the genetically modified animal may comprise at least one inactivated chromosomal sequence encoding a neurotransmission-related protein. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional neurotransmission-related protein is not produced. The inactivated chromosomal sequence comprises no exogenously introduced sequence. Such an animal may be termed a “knockout.” The inactivated chromosomal sequence comprises no exogenously introduced sequence. Also included herein are genetically modified animals in which two, three, four, five, six, seven, eight, nine, or ten or more chromosomal sequences encoding proteins associated with neurotransmission-related disorders are inactivated.

In another embodiment, the genetically modified animal may comprise at least one edited chromosomal sequence encoding an orthologous protein associated with a neurotransmission-related disorder. The edited chromosomal sequence encoding an orthologous neurotransmission-related protein may be modified such that it codes for an altered protein. For example, the edited chromosomal sequence encoding a neurotransmission-related protein may comprise at least one modification such that an altered version of the protein is produced. In some embodiments, the edited chromosomal sequence comprises at least one modification such that the altered version of the neurotransmission-related protein results in a neurotransmission-related disorder in the animal. In other embodiments, the edited chromosomal sequence encoding a neurotransmission-related protein comprises at least one modification such that the altered version of the protein protects against a neurotransmission-related disorder in the animal. The modification may be a missense mutation in which substitution of one nucleotide for another nucleotide changes the identity of the coded amino acid.

In yet another embodiment, the genetically modified animal may comprise at least one chromosomally integrated sequence. The chromosomally integrated sequence may encode an orthologous neurotransmission-related protein, an endogenous neurotransmission-related protein, or combinations of both. For example, a sequence encoding an orthologous protein or an endogenous protein may be integrated into a chromosomal sequence encoding a protein such that the chromosomal sequence is inactivated, but wherein the exogenous sequence may be expressed. In such a case, the sequence encoding the orthologous protein or endogenous protein may be operably linked to a promoter control sequence. Alternatively, a sequence encoding an orthologous protein or an endogenous protein may be integrated into a chromosomal sequence without affecting expression of a chromosomal sequence. For example, a sequence encoding a neurotransmission-related protein may be integrated into a “safe harbor” locus, such as the Rosa26 locus, HPRT locus, or AAV locus. An animal comprising a chromosomally integrated sequence encoding a neurotransmission-related protein may be called a “knock-in.” In one iteration of the disclosure, an animal comprising a chromosomally integrated sequence encoding a neurotransmission-related protein may comprise no selectable marker. The present disclosure also encompasses genetically modified animals in which two, three, four, five, six, seven, eight, nine, or ten or more sequences encoding protein(s) associated with neurotransmission-related disorders are integrated into the genome.

The chromosomally integrated sequence encoding a neurotransmission-related protein may encode the wild type form of the protein. Alternatively, the chromosomally integrated sequence encoding a neurotransmission-related protein may comprise at least one modification such that an altered version of the protein is produced. In some embodiments, the chromosomally integrated sequence encoding a neurotransmission-related protein comprises at least one modification such that the altered version of the protein produced causes a neurotransmission-related disorder. In other embodiments, the chromosomally integrated sequence encoding a neurotransmission-related protein comprises at least one modification such that the altered version of the protein protects against the development of a neurotransmission-related disorder.

In yet another embodiment, the genetically modified animal may comprise at least one edited chromosomal sequence encoding a protein associated with a neurotransmission-related disorder such that the expression pattern of the protein is altered. For example, regulatory regions controlling the expression of the protein, such as a promoter or transcription binding site, may be altered such that the protein associated with a neurotransmission-related disorder is over-produced, or the tissue-specific or temporal expression of the protein is altered, or a combination thereof.

Alternatively, the expression pattern of the protein associated with a neurotransmission-related disorder may be altered using a conditional knockout system. A non-limiting example of a conditional knockout system includes a Cre-lox recombination system. A Cre-lox recombination system comprises a Cre recombinase enzyme, a site-specific DNA recombinase that can catalyse the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a protein associated with a neurotransmission-related disorder. The genetically modified animal comprising the lox-flanked chromosomal sequence encoding a protein associated with a neurotransmission-related disorder may then be crossed with another genetically modified animal expressing Cre recombinase. Progeny animals comprising the lox-flanked chromosomal sequence and the Cre recombinase are then produced, and the lox-flanked chromosomal sequence encoding a protein associated with a neurotransmission-related disorder is recombined, leading to deletion or inversion of the chromosomal sequence encoding the protein. Expression of Cre recombinase may be temporally and conditionally regulated to effect temporally and conditionally regulated recombination of the chromosomal sequence encoding a protein associated with a neurotransmission-related disorder.

In an additional embodiment, the genetically modified animal may be a “humanized” animal comprising at least one chromosomally integrated sequence encoding a functional human neurotransmission-related protein. The functional human neurotransmission-related protein may have no corresponding ortholog in the genetically modified animal. Alternatively, the wild-type animal from which the genetically modified animal is derived may comprise an ortholog corresponding to the functional human neurotransmission-related protein. In this case, the orthologous sequence in the “humanized” animal is inactivated such that no functional protein is made and the “humanized” animal comprises at least one chromosomally integrated sequence encoding the human neurotransmission-related protein. Those of skill in the art appreciate that “humanized” animals may be generated by crossing a knock out animal with a knock in animal comprising the chromosomally integrated sequence.

### (a) Neurotransmission-Related Proteins

Neurotransmission-related proteins are a diverse set of proteins associated with susceptibility for developing a neurotransmission disorder, the presence of a neurotransmission disorder, the severity of a neurotransmission disorder or any combination thereof. Non-limiting examples of a neurotransmission disorder include amylotropic lateral sclerosis (ALS), spinocerebellar ataxias (SCA), in particular SCA2, Alzheimer's; autism, mental retardation, Rett's syndrome; fragile X syndrome, depression, schizophrenia, bi-polar disorders, disorders of learning, memory and behavior, anxiety, brain injury, seizure disorders, Huntington's disease (chorea), mania, neuroleptic malignant syndrome, pain, Parkinsonism, Parkinson's disease, tardive dyskinesia, myasthenia gravis, episodic ataxias, hyperkalemic periodic paralysis, hypokalemic periodic paralysis, Lambert-Eaton syndrome, paramyotonia congenita, Rasmussen's encephalitis, startle disease (hyperexplexia, stiff baby syndrome), and poisoning such as botulism, mushroom poisoning, organophosphates, snake venom, such as from Bungarus multicinctus (Taiwanese banded krait).

The neurotransmission-related proteins are typically selected based on an experimental association of the neurotransmission-related protein to a neurotransmission disorder. For example, the production rate or circulating concentration of a neurotransmission-related protein may be elevated or depressed in a population having a neurotransmission disorder relative to a population lacking the neurotransmission disorder. Differences in protein levels may be assessed using proteomic techniques including but not limited to Western blot, immunohistochemical staining, enzyme linked immunosorbent assay (ELISA), and mass spectrometry. Alternatively, the neurotransmission-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

Non-limiting examples of neurotransmission-related proteins include SST (somatostatin), NOS1 (nitric oxide synthase 1 (neuronal)), ADRA2A (adrenergic, alpha-2A-, receptor), ADRA2C (adrenergic, alpha-2C-, receptor), TACR1 (tachykinin receptor 1), HTR2C (5-hydroxytryptamine (serotonin) receptor 2C), SLC1A2 (solute carrier family 1 (glial high affinity glutamate transporter), member 2), GRM5 (glutamate receptor, metabotropic 5), GRM2 (glutamate receptor, metabotropic 2), GABRG3 (gamma-aminobutyric acid (GABA) A receptor, gamma 3), CACNA1B (calcium channel, voltage-dependent, N type, alpha 1B subunit), NOS2 (nitric oxide synthase 2, inducible), SLC6A5 (solute carrier family 6 (neurotransmitter transporter, glycine), member 5), GABRG1 (gamma-aminobutyric acid (GABA) A receptor, gamma 1), NOS3 (nitric oxide synthase 3 (endothelial cell)), GRM3 (glutamate receptor, metabotropic 3), HTR6 (5-hydroxytryptamine (serotonin) receptor 6), SLC1A3 (solute carrier family 1 (glial high affinity glutamate transporter), member 3), GRM7 (glutamate receptor, metabotropic 7), HRH1 (histamine receptor H1), SLC1A1 (solute carrier family 1 (neuronal/epithelial high affinity glutamate transporter, system Xag), member 1), GRM4 (glutamate receptor, metabotropic 4), GLUD2 (glutamate dehydrogenase 2), ADRA2B (adrenergic, alpha-2B-, receptor), SLC1A6 (solute carrier family 1 (high affinity aspartate/glutamate transporter), member 6), GRM6 (glutamate receptor, metabotropic 6), SLC1A7 (solute carrier family 1 (glutamate transporter), member 7), SLC6A11 (solute carrier family 6 (neurotransmitter transporter, GABA), member 11), CACNA1A (calcium channel, voltage-dependent, P/Q type, alpha 1A subunit), CACNA1 G (calcium channel, voltage-dependent, T type, alpha 1G subunit), GRM1 (glutamate receptor, metabotropic 1), CACNA1 H (calcium channel, voltage-dependent, T type, alpha 1H subunit), GRM8 (glutamate receptor, metabotropic 8), CHRNA3 (cholinergic receptor, nicotinic, alpha 3), P2RY2 (purinergic receptor P2Y, G-protein coupled, 2), TRPV6 (transient receptor potential cation channel, subfamily V, member 6), CACNA1 E (calcium channel, voltage-dependent, R type, alpha 1 E subunit), ACCN1 (amiloride-sensitive cation channel 1, neuronal), CACNA1 I (calcium channel, voltage-dependent, T type, alpha 1I subunit), GABARAP (GABA (A) receptor-associated protein), P2RY1 (purinergic receptor P2Y, G-protein coupled, 1), P2RY6 (pyrimidinergic receptor P2Y, G-protein coupled, 6), RPH3A (rabphilin 3A homolog (mouse)), HDC (histidine decarboxylase), P2RY14 (purinergic receptor P2Y, G-protein coupled, 14), P2RY4 (pyrimidinergic receptor P2Y, G-protein coupled, 4), P2RY10 (purinergic receptor P2Y, G-protein coupled, 10), SLC28A3 (solute carrier family 28 (sodium-coupled nucleoside transporter), member 3), NOSTRIN (nitric oxide synthase trafficker), P2RY13 (purinergic receptor P2Y, G-protein coupled, 13), P2RY8 (purinergic receptor P2Y, G-protein coupled, 8), P2RY11 (purinergic receptor P2Y, G-protein coupled, 11), SLC6A3 (solute carrier family 6 (neurotransmitter transporter, dopamine), member 3), HTR3A (5-hydroxytryptamine (serotonin) receptor 3A), DRD2 (dopamine receptor D2), HTR2A (5-hydroxytryptamine (serotonin) receptor 2A), TH (tyrosine hydroxylase), CNR1 (cannabinoid receptor 1 (brain)), VIP (vasoactive intestinal peptide), NPY (neuropeptide Y), GAL (galanin prepropeptide), TAC1 (tachykinin, precursor 1), SYP (synaptophysin), SLC6A4 (solute carrier family 6 (neurotransmitter transporter, serotonin), member 4), DBH (dopamine beta-hydroxylase (dopamine beta-monooxygenase)), DRD3 (dopamine receptor D3), NR3C1 (nuclear receptor subfamily 3, group C, member 1 (glucocorticoid receptor)), HTR1B (5-hydroxytryptamine (serotonin) receptor 1B), GABBR1 (gamma-aminobutyric acid (GABA) B receptor, 1), CALCA (calcitonin-related polypeptide alpha), CRH (corticotropin releasing hormone), HTR1A (5-hydroxytryptamine (serotonin) receptor 1A), TACR2 (tachykinin receptor 2), COMT (catechol-O-methyltransferase), GRIN2B (glutamate receptor, ionotropic, N-methyl D-aspartate 2B), GRIN2A (glutamate receptor, ionotropic, N-methyl D-aspartate 2A), PRL (prolactin), ACHE (acetylcholinesterase (Yt blood group)), ADRB2 (adrenergic, beta-2-, receptor, surface), ACE (angiotensin I converting enzyme (peptidyl-dipeptidase A) 1), SNAP25 (synaptosomal-associated protein, 25 kDa), GABRA5 (gamma-aminobutyric acid (GABA) A receptor, alpha 5), MECP2 (methyl CpG binding protein 2 (Rett syndrome)), BCHE (butyrylcholinesterase), ADRB1 (adrenergic, beta-1-, receptor), GABRA1 (gamma-aminobutyric acid (GABA) A receptor, alpha 1), GCH1 (GTP cyclohydrolase 1), DDC (dopa decarboxylase (aromatic L-amino acid decarboxylase)), MAOB (monoamine oxidase B), DRD5 (dopamine receptor D5), GABRE (gamma-aminobutyric acid (GABA) A receptor, epsilon), SLC6A2 (solute carrier family 6 (neurotransmitter transporter, noradrenalin), member 2), GABRR2 (gamma-aminobutyric acid (GABA) receptor, rho 2), SV2A (synaptic vesicle glycoprotein 2A), GABRR1 (gamma-aminobutyric acid (GABA) receptor, rho 1), GHRH (growth hormone releasing hormone), CCK (cholecystokinin), PDYN (prodynorphin), SLC6A9 (solute carrier family 6 (neurotransmitter transporter, glycine), member 9), KCND1 (potassium voltage-gated channel, Shal-related subfamily, member 1), SRR (serine racemase), DYT10 (dystonia 10), MAPT (microtubule-associated protein tau), APP (amyloid beta (A4) precursor protein), CTSB (cathepsin B), ADA (adenosine deaminase), AKT1 (v-akt murine thymoma viral oncogene homolog 1), GRIN1 (glutamate receptor, ionotropic, N-methyl D-aspartate 1), BDNF (brain-derived neurotrophic factor), HMOX1 (heme oxygenase (decycling) 1), OPRM1 (opioid receptor, mu 1), GRIN2C (glutamate receptor, ionotropic, N-methyl D-aspartate 2C), GRIA1 (glutamate receptor, ionotropic, AMPA 1), GABRA6 (gamma-aminobutyric acid (GABA) A receptor, alpha 6), FOS (FBJ murine osteosarcoma viral oncogene homolog), GABRG2 (gamma-aminobutyric acid (GABA) A receptor, gamma 2), GABRB3 (gamma-aminobutyric acid (GABA) A receptor, beta 3), OPRK1 (opioid receptor, kappa 1), GABRB2 (gamma-aminobutyric acid (GABA) A receptor, beta 2), GABRD (gamma-aminobutyric acid (GABA) A receptor, delta), ALDH5A1 (aldehyde dehydrogenase 5 family, member A1), GAD1 (glutamate decarboxylase 1 (brain, 67 kDa)), NSF (N-ethylmaleimide-sensitive factor), GRIN2D (glutamate receptor, ionotropic, N-methyl D-aspartate 2D), ADORA1 (adenosine A1 receptor), GABRA2 (gamma-aminobutyric acid (GABA) A receptor, alpha 2), GLRA1 (glycine receptor, alpha 1), CHRM3 (cholinergic receptor, muscarinic 3), CHAT (choline acetyltransferase), KNG1 (kininogen 1), HMOX2 (heme oxygenase (decycling) 2), DRD4 (dopamine receptor D4), MAOA (monoamine oxidase A), CHRM2 (cholinergic receptor, muscarinic 2), ADORA2A (adenosine A2a receptor), STXBP1 (syntaxin binding protein 1), GABRA3 (gamma-aminobutyric acid (GABA) A receptor, alpha 3), TPH1 (tryptophan hydroxylase 1), HCRTR1 (hypocretin (orexin) receptor 1), HCRTR2 (hypocretin (orexin) receptor 2), CHRM1 (cholinergic receptor, muscarinic 1), FOLH1 (folate hydrolase (prostate-specific membrane antigen) 1), AANAT (arylalkylamine N-acetyltransferase), INS (insulin), NR3C2 (nuclear receptor subfamily 3, group C, member 2), FAAH (fatty acid amide hydrolase), GALR2 (galanin receptor 2), ADCYAP1 (adenylate cyclase activating polypeptide 1 (pituitary)), PPP1R1 B (protein phosphatase 1, regulatory (inhibitor) subunit 1B), HOMER1 (homer homolog 1 (Drosophila)), ADCY10 (adenylate cyclase 10 (soluble)), PSEN2 (presenilin 2 (Alzheimer disease 4)), UBE3A (ubiquitin protein ligase E3A), SOD1 (superoxide dismutase 1, soluble), LYN (v-yes-1 Yamaguchi sarcoma viral related oncogene homolog), TSC2 (tuberous sclerosis 2), PRKCA (protein kinase C, alpha), PPARG (peroxisome proliferator-activated receptor gamma), ESR1 (estrogen receptor 1), NTRK1 (neurotrophic tyrosine kinase, receptor, type 1), EGFR (epidermal growth factor receptor (erythroblastic leukemia viral (v-erb-b) oncogene homolog, avian)), S100B (S100 calcium binding protein B), NTRK3 (neurotrophic tyrosine kinase, receptor, type 3), PLCG2 (phospholipase C, gamma 2 (phosphatidylinositol-specific)), NTRK2 (neurotrophic tyrosine kinase, receptor, type 2), DNMT1 (DNA (cytosine-5-)-methyltransferase 1), EGF (epidermal growth factor (beta-urogastrone)), GRIA3 (glutamate receptor, ionotrophic, AMPA 3), NCAM1 (neural cell adhesion molecule 1), CDKN1A (cyclin-dependent kinase inhibitor 1A (p21, Cip1)), BCL2L1 (BCL2-like 1), TP53 (tumor protein p53), CASP9 (caspase 9, apoptosis-related cysteine peptidase), CCKBR (cholecystokinin B receptor), PARK2 (Parkinson's disease (autosomal recessive, juvenile) 2, parkin), ADRA1B (adrenergic, alpha-1B-, receptor), CASP3 (caspase 3, apoptosis-related cysteine peptidase), PRNP (prion protein), CRHR1 (corticotropin releasing hormone receptor 1), L1 CAM (L1 cell adhesion molecule), NGFR (nerve growth factor receptor (TNFR superfamily, member 16)), CREB1 (cAMP responsive element binding protein 1), PLCG1 (phospholipase C, gamma 1), CAV1 (caveolin 1, caveolae protein, 22 kDa), ABCC8 (ATP-binding cassette, sub-family C (CFTR/MRP), member 8), ACTN2 (actinin, alpha 2), GRIA2 (glutamate receptor, ionotropic, AMPA 2), HPRT1 (hypoxanthine phosphoribosyltransferase 1), SYN1 (synapsin I), CSNK2A1 (casein kinase 2, alpha 1 polypeptide), GRIK1 (glutamate receptor, ionotropic, kainate 1), ABCB1 (ATP-binding cassette, sub-family B (MDR/TAP), member 1), AVPR2 (arginine vasopressin receptor 2), HTR4 (5-hydroxytryptamine (serotonin) receptor 4), C3 (complement component 3), AGT (angiotensinogen (serpin peptidase inhibitor, clade A, member 8)), AGTR1 (angiotensin II receptor, type 1), CDK5 (cyclin-dependent kinase 5), LRP1 (low density lipoprotein receptor-related protein 1), ARRB2 (arrestin, beta 2), PLD2 (phospholipase D2), OPRD1 (opioid receptor, delta 1), GNB3 (guanine nucleotide binding protein (G protein), beta polypeptide 3), PIK3CG (phosphoinositide-3-kinase, catalytic, gamma polypeptide), APAF1 (apoptotic peptidase activating factor 1), SSTR2 (somatostatin receptor 2), IL2 (interleukin 2), ADORA3 (adenosine A3 receptor), ADRA1A (adrenergic, alpha-1A-, receptor), HTR7 (5-hydroxytryptamine (serotonin) receptor 7 (adenylate cyclase-coupled)), ADRBK2 (adrenergic, beta, receptor kinase 2), ALOX5 (arachidonate 5-lipoxygenase), NPR1 (natriuretic peptide receptor A/guanylate cyclase A (atrionatriuretic peptide receptor A)), AVPR1A (arginine vasopressin receptor 1A), CHRNB1 (cholinergic receptor, nicotinic, beta 1 (muscle)), SET (SET nuclear oncogene), PAH (phenylalanine hydroxylase), POMC (proopiomelanocortin), LEPR (leptin receptor), SDC2 (syndecan 2), VIPR1 (vasoactive intestinal peptide receptor 1), DBI (diazepam binding inhibitor (GABA receptor modulator, acyl-Coenzyme A binding protein)), NPY1 R (neuropeptide Y receptor Y1), NPR2 (natriuretic peptide receptor B/guanylate cyclase B (atrionatriuretic peptide receptor B)), CNR2 (cannabinoid receptor 2 (macrophage)), LEP (leptin), CCKAR (cholecystokinin A receptor), GLRB (glycine receptor, beta), KCNQ2 (potassium voltage-gated channel, KQT-like subfamily, member 2), CHRNA2 (cholinergic receptor, nicotinic, alpha 2 (neuronal)), BDKRB2 (bradykinin receptor B2), CHRNA1 (cholinergic receptor, nicotinic, alpha 1 (muscle)), CHRND (cholinergic receptor, nicotinic, delta), CHRNA7 (cholinergic receptor, nicotinic, alpha 7), PLD1 (phospholipase D1, phosphatidylcholine-specific), NRXN1 (neurexin 1), NRP1 (neuropilin 1), DLG3 (discs, large homolog 3 (Drosophila)), GNAQ (guanine nucleotide binding protein (G protein), q polypeptide), DRD1 (dopamine receptor D1), PRKG1 (protein kinase, cGMP-dependent, type I), CNTNAP2 (contactin associated protein-like 2), EDN3 (endothelin 3), ABAT (4-aminobutyrate aminotransferase), TDO2 (tryptophan 2,3-dioxygenase), NEUROD1 (neurogenic differentiation 1), CHRNE (cholinergic receptor, nicotinic, epsilon), CHRNB2 (cholinergic receptor, nicotinic, beta 2 (neuronal)), CHRNB3 (cholinergic receptor, nicotinic, beta 3), HTR1 D (5-hydroxytryptamine (serotonin) receptor 1 D), ADRA1 D (adrenergic, alpha-1 D-, receptor), HTR2B (5-hydroxytryptamine (serotonin) receptor 2B), GRIK3 (glutamate receptor, ionotropic, kainate 3), NPY2R (neuropeptide Y receptor Y2), GRIK5 (glutamate receptor, ionotropic, kainate 5), GRIA4 (glutamate receptor, ionotrophic, AMPA 4), EDN1 (endothelin 1), PRLR (prolactin receptor), GABRB1 (gamma-aminobutyric acid (GABA) A receptor, beta 1), GARS (glycyl-tRNA synthetase), GRIK2 (glutamate receptor, ionotropic, kainate 2), ALOX12 (arachidonate 12-lipoxygenase), GAD2 (glutamate decarboxylase 2 (pancreatic islets and brain, 65 kDa)), LHCGR (luteinizing hormone/choriogonadotropin receptor), SHMT1 (serine hydroxymethyltransferase 1 (soluble)), PDXK (pyridoxal (pyridoxine, vitamin B6) kinase), LIF (leukemia inhibitory factor (cholinergic differentiation factor)), PLCD1 (phospholipase C, delta 1), NTF3 (neurotrophin 3), NFE2L2 (nuclear factor (erythroid-derived 2)-like 2), PLCB4 (phospholipase C, beta 4), GNRHR (gonadotropin-releasing hormone receptor), NLGN1 (neuroligin 1), PPP2R4 (protein phosphatase 2A activator, regulatory subunit 4), SSTR3 (somatostatin receptor 3), CRHR2 (corticotropin releasing hormone receptor 2), NGF (nerve growth factor (beta polypeptide)), NRCAM (neuronal cell adhesion molecule), NRXN3 (neurexin 3), GNRH1 (gonadotropin-releasing hormone 1 (luteinizing-releasing hormone)), TRHR (thyrotropin-releasing hormone receptor), ARRB1 (arrestin, beta 1), INPP1 (inositol polyphosphate-1-phosphatase), PTN (pleiotrophin), PSMD10 (proteasome (prosome, macropain) 26S subunit, non-ATPase, 10), DLG1 (discs, large homolog 1 (Drosophila)), PSMB8 (proteasome (prosome, macropain) subunit, beta type, 8 (large multifunctional peptidase 7)), CYCS (cytochrome c, somatic), ADORA2B (adenosine A2b receptor), ADRB3 (adrenergic, beta-3-, receptor), CHGA (chromogranin A (parathyroid secretory protein 1)), ADM (adrenomedullin), GABRP (gamma-aminobutyric acid (GABA) A receptor, pi), GLRA2 (glycine receptor, alpha 2), PRKG2 (protein kinase, cGMP-dependent, type II), GLS (glutaminase), TACR3 (tachykinin receptor 3), ALDH7A1 (aldehyde dehydrogenase 7 family, member A1), GABBR2 (gamma-aminobutyric acid (GABA) B receptor, 2), GDNF (glial cell derived neurotrophic factor), CNTFR (ciliary neurotrophic factor receptor), CNTN2 (contactin 2 (axonal)), TOR1A (torsin family 1, member A (torsin A)), CNTN1 (contactin 1), CAMK1 (calcium/calmodulin-dependent protein kinase I), NPPB (natriuretic peptide precursor B), OXTR (oxytocin receptor), OSM (oncostatin M), VIPR2 (vasoactive intestinal peptide receptor 2), CHRNB4 (cholinergic receptor, nicotinic, beta 4), CHRNA5 (cholinergic receptor, nicotinic, alpha 5), AVP (arginine vasopressin), RELN (reelin), GRLF1 (glucocorticoid receptor DNA binding factor 1), NPR3 (natriuretic peptide receptor C/guanylate cyclase C (atrionatriuretic peptide receptor C)), GRIK4 (glutamate receptor, ionotropic, kainate 4), KISS1 (KiSS-1 metastasis-suppressor), HTR5A (5-hydroxytryptamine (serotonin) receptor 5A), ADCYAP1 R1 (adenylate cyclase activating polypeptide 1 (pituitary) receptor type I), GABRA4 (gamma-aminobutyric acid (GABA) A receptor, alpha 4), GLRA3 (glycine receptor, alpha 3), INHBA (inhibin, beta A), DLG2 (discs, large homolog 2 (Drosophila)), PPYR1 (pancreatic polypeptide receptor 1), SSTR4 (somatostatin receptor 4), NPPA (natriuretic peptide precursor A), SNAP23 (synaptosomal-associated protein, 23 kDa), AKAP9 (A kinase (PRKA) anchor protein (yotiao) 9), NRXN2 (neurexin 2), FHL2 (four and a half LIM domains 2), TJP1 (tight junction protein 1 (zona occludens 1)), NRG1 (neuregulin 1), CAMK4 (calcium/calmodulin-dependent protein kinase IV), CAV3 (caveolin 3), VAMP2 (vesicle-associated membrane protein 2 (synaptobrevin 2)), GALR1 (galanin receptor 1), GHRHR (growth hormone releasing hormone receptor), HTR1 E (5-hydroxytryptamine (serotonin) receptor 1 E), PENK (proenkephalin), HTT (huntingtin), HOXA1 (homeobox A1), NPY5R (neuropeptide Y receptor Y5), UNC119 (unc-119 homolog (C. elegans)), TAT (tyrosine aminotransferase), CNTF (ciliary neurotrophic factor), SHMT2 (serine hydroxymethyltransferase 2 (mitochondrial)), ENTPD1 (ectonucleoside triphosphate diphosphohydrolase 1), GRIP1 (glutamate receptor interacting protein 1), GRP (gastrin-releasing peptide), NCAM2 (neural cell adhesion molecule 2), SSTR1 (somatostatin receptor 1), CLTB (clathrin, light chain (Lcb)), DAO (D-amino-acid oxidase), QDPR (quinoid dihydropteridine reductase), PYY (peptide YY), PNMT (phenylethanolamine N-methyltransferase), NTSR1 (neurotensin receptor 1 (high affinity)), NTS (neurotensin), HCRT (hypocretin (orexin) neuropeptide precursor), SNAP29 (synaptosomal-associated protein, 29 kDa), SNAP91 (synaptosomal-associated protein, 91 kDa homolog (mouse)), MADD (MAP-kinase activating death domain), IDO1 (indoleamine 2,3-dioxygenase 1), TPH2 (tryptophan hydroxylase 2), TAC3 (tachykinin 3), GRIN3A (glutamate receptor, ionotropic, N-methyl-D-aspartate 3A), REN (renin), GALR3 (galanin receptor 3), MAGI2 (membrane associated guanylate kinase, WW and PDZ domain containing 2), KCNJ9 (potassium inwardly-rectifying channel, subfamily J, member 9), BDKRB1 (bradykinin receptor B1), CHRNA6 (cholinergic receptor, nicotinic, alpha 6), CHRM5 (cholinergic receptor, muscarinic 5), CHRNG (cholinergic receptor, nicotinic, gamma), SLC6A1 (solute carrier family 6 (neurotransmitter transporter, GABA), member 1), ENTPD2 (ectonucleoside triphosphate diphosphohydrolase 2), CALCB (calcitonin-related polypeptide beta), SHBG (sex hormone-binding globulin), SERPINA6 (serpin peptidase inhibitor, clade A (alpha-1 antiproteinase, antitrypsin), member 6), NRG2 (neuregulin 2), PNOC (prepronociceptin), NAPA (N-ethylmaleimide-sensitive factor attachment protein, alpha), PICK1 (protein interacting with PRKCA 1), PLCD4 (phospholipase C, delta 4), GCDH (glutaryl-Coenzyme A dehydrogenase), NLGN2 (neuroligin 2), NBEA (neurobeachin), ATP10A (ATPase, class V, type 10A), RAPGEF4 (Rap guanine nucleotide exchange factor (GEF) 4), UCN (urocortin), PCSK6 (proprotein convertase subtilisin/kexin type 6), HTR1 F (5-hydroxytryptamine (serotonin) receptor 1F), SGCB (sarcoglycan, beta (43 kDa dystrophin-associated glycoprotein)), GABRQ (gamma-aminobutyric acid (GABA) receptor, theta), GHRL (ghrelin/obestatin prepropeptide), NCALD (neurocalcin delta), NEUROD2 (neurogenic differentiation 2), DPEP1 (dipeptidase 1 (renal)), SLC1A4 (solute carrier family 1 (glutamate/neutral amino acid transporter), member 4), DNM3 (dynamin 3), SLC6A12 (solute carrier family 6 (neurotransmitter transporter, betaine/GABA), member 12), SLC6A6 (solute carrier family 6 (neurotransmitter transporter, taurine), member 6), YME1L1 (YME1-like 1 (S. cerevisiae)), VSNL1 (visinin-like 1), SLC17A7 (solute carrier family 17 (sodium-dependent inorganic phosphate cotransporter), member 7), HOMER2 (homer homolog 2 (Drosophila)), SYT7 (synaptotagmin VII), TFIP11 (tuftelin interacting protein 11), GMFB (glia maturation factor, beta), PREB (prolactin regulatory element binding), NTSR2 (neurotensin receptor 2), NTF4 (neurotrophin 4), PPP1R9B (protein phosphatase 1, regulatory (inhibitor) subunit 9B), DISC1 (disrupted in schizophrenia 1), NRG3 (neuregulin 3), OXT (oxytocin, prepropeptide), TRH (thyrotropin-releasing hormone), NISCH (nischarin), CRHBP (corticotropin releasing hormone binding protein), SLC6A13 (solute carrier family 6 (neurotransmitter transporter, GABA), member 13), NPPC (natriuretic peptide precursor C), CNTN3 (contactin 3 (plasmacytoma associated)), KAT5 (K (lysine) acetyltransferase 5), CNTN6 (contactin 6), KIAA0101 (KIAA0101), PANX1 (pannexin 1), CTSL1 (cathepsin L1), EARS2 (glutamyl-tRNA synthetase 2, mitochondrial (putative)), CRIPT (cysteine-rich PDZ-binding protein), CORT (cortistatin), DLGAP4 (discs, large (Drosophila) homolog-associated protein 4), ASTN2 (astrotactin 2), HTR3B (5-hydroxytryptamine (serotonin) receptor 3B), PMCH (pro-melanin-concentrating hormone), TSPO (translocator protein (18 kDa)), GDF2 (growth differentiation factor 2), CNTNAP1 (contactin associated protein 1), GNRH2 (gonadotropin-releasing hormone 2), AUTS2 (autism susceptibility candidate 2), SV2C (synaptic vesicle glycoprotein 2C), CARTPT (CART prepropeptide), NSUN4 (NOP2/Sun domain family, member 4), CNTN5 (contactin 5), NEUROD4 (neurogenic differentiation 4), NEUROG1 (neurogenin 1), SLTM (SAFB-like, transcription modulator), GNRHR2 (gonadotropin-releasing hormone (type 2) receptor 2), ASTN1 (astrotactin 1), SLC22A18 (solute carrier family 22, member 18), SLC17A6 (solute carrier family 17 (sodium-dependent inorganic phosphate cotransporter), member 6), GABRR3 (gamma-aminobutyric acid (GABA) receptor, rho 3), DAOA (D-amino acid oxidase activator), ENSG00000123384, nd NOS2P1 (nitric oxide synthase 2 pseudogene 1).

Exemplary neurotransmission-related proteins include 5-HTT (5-hydroxyltryptamine transporter), (SLC6A4) (Solute carrier family 6, member 4), COMT (Catechol-O-methyltransferase), DRD (DRD1A) (Dopamine receptor D1A), SLC6A3 (Solute carrier family 6, member 3), DAO (DAO1) (D-amino-acid oxidase), DTNBP1 (Dystrobrevin binding protein 1) and any combination thereof.

(i) 5-HTT

5-HTT, also known as 5-5-hydroxyltryptamine transporter, and as SLC6A4 or solute carrier family 6 (neurotransmitter transporter, serotonin), member 4, and as hSERT, HTT, 5-HTTLPR, OCD1 and SERT, is an integral membrane protein that in humans is encoded by the SLC6A4 gene. 5-HTT is found widely distributed throughout the brain. It transports the neurotransmitter serotonin from synaptic spaces into presynaptic neurons. The encoded protein terminates the action of serotonin and recycles it in a sodium-dependent manner. This protein is a target of psychomotor stimulants, such as amphetamines and cocaine, and is a member of the sodium:neurotransmitter symporter family. A repeat length polymorphism in the promoter of this gene has been shown to affect the rate of serotonin uptake and may play a role in sudden infant death syndrome, aggressive behavior in Alzheimer disease patients, and depression-susceptibility in people experiencing emotional trauma.

(ii) COMT

COMT, also known as catechol-O-methyltransferase, is an enzyme that is encoded by the COMT gene. Two versions of this enzyme are made from the gene, a longer form, membrane-bound catechol-O-methyltransferase (MB-COMT), and a shorter form, soluble catechol-O-methyltransferase (S-COMT). In the nervous system, COMPT is localized to postsynaptic neurons where it degrades neurotransmitters such as dopamine, epinephrine and norepinephrine following their release. COMT is also important in the metabolism of catechol drugs used in the treatment of hypertension, asthma and Parkinson's disease. Loss of one copy of the COMT gene, which occurs in the 22q11.2 deletion syndrome, is thought to be associated with increased risk of behavioral problems and mental illness. Polymorphic variations in the COMT gene, such as replacement of valine with methionine at position 158 in MB-COMT and at position 108 in S-COMT (known as Val 108/158 polymorphism) have been studied and may be associated with schizophrenia. The Val 108/158 polymorphism has also been studied as a risk factor for other disorders that affect cognition and emotion, such as bipolar disorder, panic disorder, anxiety, obsessive-compulsive disorder (OCD), eating disorders and attention deficit hyperactivity disorder (ADHD).

(iii) DRD

DRD1, also known as DRD1A or dopamine D1 receptor, and as DADR, is a protein that is encoded by the DRDlgene. DRD1A is the D1 subtype of dopamine receptor, which is the most abundant dopamine receptor in the central nervous system (CNS). D1 receptors are widely expressed throughout the brain. D1 receptors regulate neuronal growth and development, mediate some behavioral responses, and modulate dopamine receptor D2-mediated events. Dysfunction of dopaminagenic neurotransmission in the CNS has been implicated in a variety of neuropsychiatric disorders, including social phobia, Tourette's syndrome, Parkinson's disease, schizophrenia, neuroleptic malignant syndrome, ADHD and drug and alcohol dependence.

(iv) SLC6A3

SLC6A3, also known as solute carrier family 6 (neurotransmitter transporter, dopamine), member 3, and as DAT or dopamine active transporter, and as DAT1, is an integral membrane protein that is encoded by the SLC6A3 gene. It is widely distributed throughout the brain in areas of dopaminergic activity. SLC6A3 provides rapid clearance of dopamine, epinephrine and norepinephrine form the synaptic cleft, terminating the neurotransmitter signal. There is a variable number tandem repeat or VNTR at the 3′ end of this gene, which can be present in 3 to 11 copies. Variation in the number of repeats has been associated with idiopathic epilepsy, ADHD, alcohol and cocaine dependence, susceptibility to Parkinson's disease and protection against nicotine dependence. There is also evidence of the role of dopamine transporters in bipolar disorder and clinical depression.

(v) DAO

DAO, also known as D-amino-acid oxidase, and as DAMOX, DAAO, MGC35381, OXDA and EC 1.4.3.3, is a peroxisomal enzyme encoded by the DAO gene. DAO regulates the level of the neuromodulator D-serine in the brain and contributes to dopamine synthesis. Post mortem studies in humans have found two-fold elevated levels of DAO associated with schizophrenia. DAO may act as a detoxifying agent which removes D-amino acids that accumulate during aging.

(vi) DTNBP1

DTNBP1, also known as dystrobrevin binding protein 1, and as HPS7, My031, DBND, Hermansky-Pudlak syndrome 7 protein, Dysbindin, MGC20210, DKFZp564K192, FLJ30031, and SDY, is a protein encoded by the DTNBP1 gene. It is expressed in many tissues, including neuronal tissue of the brain, particularly in axon bundles. DTNBP1 may play a role in organelle biogenesis associated with melanosomes, platelet dense granules and lysosomes. It plays a role in intracellular vesicle trafficking, synaptic vesicle trafficking and neurotransmitter release. It may be required for normal dopamine homeostasis in the cerebral cortex, hippocampus and hypothalamus. It plays a role in the regulation of cell surface exposure of DRD2 and contributes to the regulation of dopamine signaling. It may also play a role in actin cytoskeleton reorganization and neurite outgrowth and may modulate MAPK8 phosphorylation. Mutations in DTNBP1 are associated with Hermansky-Pudlak syndrome type 7. This gene may be associated with schizophrenia.

The identity of the neurotransmission-related protein whose chromosomal sequence is edited can and will vary. In general, the neurotransmission-related protein whose chromosomal sequence is edited may be 5-HTT, COMT, DRD, SLC6A3, DAO, DTNBP1, GABAa, NMDA, NMDAR, NR1, NR2a, NR2b, mGLUR1, mGLUR2, mGLUR3, mGLUR5, GLUR1, GLUR2, GAD67, GAT1, TCF4, NPAS3, GR1 K4, COMT, MAO, DBH, TyrH, CB!, CB2, FAAH, and/or MAGL. Exemplary genetically modified animals may comprise one, two, three, four, five, six, seven, eight, or nine or more inactivated chromosomal sequences encoding a neurotransmission-related protein and zero, one, two, three, four, five, six, seven or eight or more chromosomally integrated sequences encoding orthologous neurotransmission-related proteins. Table A lists preferred combinations of inactivated chromosomal sequences and integrated sequences. For example, those rows having no entry in the “Protein Sequence” column indicate a genetically modified animal in which the sequence specified in that row under “Activated Sequence” is inactivated (i.e., a knock-out). Subsequent rows indicate single or multiple knock-outs with knock-ins of one or more integrated orthologous sequences, as indicated in the “Protein Sequence” column.

TABLE A

| Activated Sequence | Protein Sequence |
|---|---|
| 5-htt | none |
| comt | none |
| drd | none |
| slc6a3 | none |
| dao | none |
| dtnbp1 | none |
| 5-htt,comt | 5-HTT,COMT |
| 5-htt,drd | 5-HTT,DRD |
| 5-htt,slc6a3 | 5-HTT,SLC6A3 |
| 5-htt,dao | 5-HTT,DAO |
| 5-htt,dtnbp1 | 5-HTT,DTNBP1 |
| comt,drd | COMT,DRD |
| comt,slc6a3 | COMT,SLC6A3 |
| comt,dao | COMT,DAO |
| comt,dtnbp1 | COMT,DTNBP1 |
| drd,slc6a3 | DRD,SLC6A3 |
| drd,dao | DRD,DAO |
| drd,dtnbp1 | DRD,DTNBP1 |
| slc6a3,dao | SLC6A3,DAO |
| slc6a3,dtnbp1 | SLC6A3,DTNBP1 |
| dao,dtnbp1 | DAO,DTNBP1 |
| 5-htt,comt,drd | 5-HTT,COMT,DRD |
| 5-htt,comt,slc6a3 | 5-HTT,COMT,SLC6A3 |
| 5-htt,comt,dao | 5-HTT,COMT,DAO |
| 5-htt,comt,dtnbp1 | 5-HTT,COMT,DTNBP1 |
| 5-htt,drd,slc6a3 | 5-HTT,DRD,SLC6A3 |
| 5-htt,drd,dao | 5-HTT,DRD,DAO |
| 5-htt,drd,dtnbp1 | 5-HTT,DRD,DTNBP1 |
| 5-htt,slc6a3,dao | 5-HTT,SLC6A3,DAO |
| 5-htt,slc6a3,dtnbp1 | 5-HTT,SLC6A3,DTNBP1 |
| 5-htt,dao,dtnbp1 | 5-HTT,DAO,DTNBP1 |
| comt,drd,slc6a3 | COMT,DRD,SLC6A3 |
| comt,drd,dao | COMT,DRD,DAO |
| comt,drd,dtnbp1 | COMT,DRD,DTNBP1 |
| comt,slc6a3,dao | COMT,SLC6A3,DAO |
| comt,slc6a3,dtnbp1 | COMT,SLC6A3,DTNBP1 |
| comt,dao,dtnbp1 | COMT,DAO,DTNBP1 |
| drd,slc6a3,dao | DRD,SLC6A3,DAO |
| drd,slc6a3,dtnbp1 | DRD,SLC6A3,DTNBP1 |
| drd,dao,dtnbp1 | DRD,DAO,DTNBP1 |
| slc6a3,dao,dtnbp1 | SLC6A3,DAO,DTNBP1 |
| 5-htt,comt,drd,slc6a3 | 5-HTT,COMT,DRD,SLC6A3 |
| 5-htt,comt,drd,dao | 5-HTT,COMT,DRD,DAO |
| 5-htt,comt,drd,dtnbp1 | 5-HTT,COMT,DRD,DTNBP1 |
| 5-htt,comt,slc6a3,dao | 5-HTT,COMT,SLC6A3,DAO |
| 5-htt,comt,slc6a3,dtnbp1 | 5-HTT,COMT,SLC6A3,DTNBP1 |
| 5-htt,comt,dao,dtnbp1 | 5-HTT,COMT,DAO,DTNBP1 |
| 5-htt,drd,slc6a3,dao | 5-HTT,DRD,SLC6A3,DAO |
| 5-htt,drd,slc6a3,dtnbp1 | 5-HTT,DRD,SLC6A3,DTNBP1 |
| 5-htt,drd,dao,dtnbp1 | 5-HTT,DRD,DAO,DTNBP1 |
| 5-htt,slc6a3,dao,dtnbp1 | 5-HTT,SLC6A3,DAO,DTNBP1 |
| comt,drd,slc6a3,dao | COMT,DRD,SLC6A3,DAO |
| comt,drd,slc6a3,dtnbp1 | COMT,DRD,SLC6A3,DTNBP1 |
| comt,drd,dao,dtnbp1 | COMT,DRD,DAO,DTNBP1 |
| comt,slc6a3,dao,dtnbp1 | COMT,SLC6A3,DAO,DTNBP1 |
| drd,slc6a3,dao,dtnbp1 | DRD,SLC6A3,DAO,DTNBP1 |
| 5-htt,comt,drd,slc6a3,dao | 5-HTT,COMT,DRD,SLC6A3,DAO |
| 5-htt,comt,drd,slc6a3,dtnbp1 | 5-HTT,COMT,DRD,SLC6A3, |
| | DTNBP1 |
| 5-htt,comt,drd,dao,dtnbp1 | 15-HTT,COMT,DRD,DAO,DTNBP1 |
| 5-htt,comt,slc6a3,dao,dtnbp1 | 5-HTT,COMT,SLC6A3,DAO, |
| | DTNBP1 |
| 5-htt,drd,slc6a3,dao,dtnbp1 | 5-HTT,DRD,SLC6A3,DAO,DTNBP1 |
| comt,drd,slc6a3,dao,dtnbp1 | COMT,DRD,SLC6A3,DAO,DTNBP1 |
| 5-htt,comt,drd,slc6a3,dao, | 5-HTT,COMT,DRD,SLC6A3,DAO, |
| dtnbp1 | DTNBP1 |

(b) animals

The term “animal,” as used herein, refers to a non-human animal. The animal may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of suitable rat strains include Dahl Salt-Sensitive, Fischer 344, Lewis, Long Evans Hooded, Sprague-Dawley, and Wistar. In another iteration of the invention, the animal does not comprise a genetically modified mouse. In each of the foregoing iterations of suitable animals for the invention, the animal does not include exogenously introduced, randomly integrated transposon sequences.

(c) neurotransmission-related protein

The neurotransmission-related protein may be from any of the animals listed above. Furthermore, the neurotransmission-related protein may be a human neurotransmission-related protein. Additionally, the neurotransmission-related protein may be a bacterial, fungal, or plant neurotransmission-related protein. The type of animal and the source of the protein can and will vary. The protein may be endogenous or exogenous (such as an orthologous protein). As an example, the genetically modified animal may be a rat, cat, dog, or pig, and the orthologous neurotransmission-related protein may be human. Alternatively, the genetically modified animal may be a rat, cat, or pig, and the orthologous neurotransmission-related protein may be canine. One of skill in the art will readily appreciate that numerous combinations are possible.

Additionally, the neurotransmission-related protein encoding gene may be modified to include a tag or reporter gene or genes as are well-known. Reporter genes include those encoding selectable markers such as cloramphenicol acetyltransferase (CAT) and neomycin phosphotransferase (neo), and those encoding a fluorescent protein such as green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

### (II) Genetically Modified Cells

A further aspect of the present disclosure provides genetically modified cells or cell lines comprising at least one edited chromosomal sequence encoding a neurotransmission-related protein. The genetically modified cell or cell line may be derived from any of the genetically modified animals disclosed herein. Alternatively, the chromosomal sequence coding a neurotransmission-related protein may be edited in a cell as detailed below. The disclosure also encompasses a lysate of said cells or cell lines.

In general, the cells will be eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CVI-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TRI cells. For an extensive list of mammalian cell lines, those of ordinary skill in the art may refer to the American Type Culture Collection catalog (ATCC®, Mamassas, Va.).

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

### (III) Zinc Finger-Mediated Genome Editing

In general, the genetically modified animal or cell detailed above in sections (I) and (II), respectively, is generated using a zinc finger nuclease-mediated genome editing process. The process for editing a chromosomal sequence comprises: (a) introducing into an embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method are described in more detail below.

(a) zinc finger nuclease

The method comprises, in part, introducing into an embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

(i) zinc finger binding domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., 51 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a FokI cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from Ito K in one cleavage monomer to produce an engineered cleavage monomer designated “E490K:1538K” and by mutating positions 486 from Q to E and 499 from Ito L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

(b) optional donor polynucleotide

The method for editing chromosomal sequences encoding neurotransmission-related proteins may further comprise introducing at least one donor polynucleotide comprising a sequence encoding a neurotransmission-related protein into the embryo or cell. A donor polynucleotide comprises at least three components: the sequence coding the neurotransmission-related protein, an upstream sequence, and a downstream sequence. The sequence encoding the protein is flanked by the upstream and downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide comprising the sequence encoding a neurotransmission-related protein may be a BAC.

The sequence of the donor polynucleotide that encodes the neurotransmission-related protein may include coding (i.e., exon) sequence, as well as intron sequences and upstream regulatory sequences (such as, e.g., a promoter). Depending upon the identity and the source of the neurotransmission-related protein, the size of the sequence encoding the neurotransmission-related protein can and will vary. For example, the sequence encoding the neurotransmission-related protein may range in size from about 1 kb to about 5,000 kb.

The donor polynucleotide also comprises upstream and downstream sequence flanking the sequence encoding the neurotransmission-related protein. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 by to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 by to about 2000 bp, about 600 by to about 1000 bp, or more particularly about 700 by to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for integrating a sequence encoding the neurotransmission-related protein, a double stranded break introduced into the chromosomal sequence by the zinc finger nuclease is repaired, via homologous recombination with the donor polynucleotide, such that the sequence encoding the neurotransmission-related protein is integrated into the chromosome. The presence of a double-stranded break facilitates integration of the sequence into the chromosome. A donor polynucleotide may be physically integrated or, alternatively, the donor polynucleotide may be used as a template for repair of the break, resulting in the introduction of the sequence encoding the neurotransmission-related protein as well as all or part of the upstream and downstream sequences of the donor polynucleotide into the chromosome. Thus, endogenous chromosomal sequence may be converted to the sequence of the donor polynucleotide.

(c) optional exchange polynucleotide

The method for editing chromosomal sequences encoding a neurotransmission-related protein may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may have at least about 80, 81, 82, 83, 84, 85, 86, 87, 88, 89, 90, 91, 92, 93, 94, 95, 96, 97, 98, or 99% sequence identity with a portion of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 by to about 10,000 by in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 by in length. In other embodiments, the sequence in the exchange polynucleotide may be about 5500, 6000, 6500, 6000, 6500, 7000, 7500, 8000, 8500, 9000, 9500, or 10,000 by in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

(d) delivery of nucleic acids

To mediate zinc finger nuclease genomic editing, at least one nucleic acid molecule encoding a zinc finger nuclease and, optionally, at least one exchange polynucleotide or at least one donor polynucleotide are delivered to the embryo or the cell of interest. Typically, the embryo is a fertilized one-cell stage embryo of the species of interest.

Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and a donor (or exchange) polynucleotide are introduced into an embryo or cell, the ratio of donor (or exchange) polynucleotide to nucleic acid encoding a zinc finger nuclease may range from about 1:10 to about 10:1. In various embodiments, the ratio of donor (or exchange) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one donor (or exchange) polynucleotide are introduced into an embryo or cell, the nucleic acids may be introduced simultaneously or sequentially. For example, nucleic acids encoding the zinc finger nucleases, each specific for a distinct recognition sequence, as well as the optional donor (or exchange) polynucleotides, may be introduced at the same time. Alternatively, each nucleic acid encoding a zinc finger nuclease, as well as the optional donor (or exchange) polynucleotides, may be introduced sequentially

(e) culturing the embryo or cell

The method of inducing genomic editing with a zinc finger nuclease further comprises culturing the embryo or cell comprising the introduced nucleic acid(s) to allow expression of the zinc finger nuclease. An embryo may be cultured in vitro (e.g., in cell culture). Typically, the embryo is cultured at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal would comprise the edited chromosomal sequence encoding the neurotransmission-related protein in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no donor (or exchange) polynucleotide, the zinc finger nuclease recognizes, binds, and cleaves the target sequence in the chromosomal sequence of interest. The double-stranded break introduced by the zinc finger nuclease is repaired by an error-prone non-homologous end-joining DNA repair process. Consequently, a deletion, insertion, or nonsense mutation may be introduced in the chromosomal sequence such that the sequence is inactivated.

In cases in which the embryo or cell comprises an expressed zinc finger nuclease as well as a donor (or exchange) polynucleotide, the zinc finger nuclease recognizes, binds, and cleaves the target sequence in the chromosome. The double-stranded break introduced by the zinc finger nuclease is repaired, via homologous recombination with the donor (or exchange) polynucleotide, such that the sequence in the donor polynucleotide is integrated into the chromosomal sequence (or a portion of the chromosomal sequence is converted to the sequence in the exchange polynucleotide). As a consequence, a sequence may be integrated into the chromosomal sequence (or a portion of the chromosomal sequence may be modified).

The genetically modified animals disclosed herein may be crossbred to create animals comprising more than one edited chromosomal sequence or to create animals that are homozygous for one or more edited chromosomal sequences. For example, two animals comprising the same edited chromosomal sequence may be crossbred to create an animal homozygous for the edited chromosomal sequence. Alternatively, animals with different edited chromosomal sequences may be crossbred to create an animal comprising both edited chromosomal sequences.

For example, animal A comprising an inactivated 5-htt chromosomal sequence may be crossed with animal B comprising a chromosomally integrated sequence encoding a human 5-HTT protein to give rise to a “humanized” 5-HTT offspring comprising both the inactivated 5-htt chromosomal sequence and the chromosomally integrated human 5-HTT sequence. Similarly, an animal comprising an inactivated 5-htt drd chromosomal sequence may be crossed with an animal comprising a chromosomally integrated sequence encoding the human neurotransmission-related DRD protein to generate “humanized” neurotransmission-related DRD offspring. Moreover, a humanized DRD animal may be crossed with a humanized 5-HTT animal to create a humanized DRD/5-HTT. Those of skill in the art will appreciate that many combinations are possible. Exemplary combinations are presented above in Table A.

In other embodiments, an animal comprising an edited chromosomal sequence disclosed herein may be crossbred to combine the edited chromosomal sequence with other genetic backgrounds. By way of non-limiting example, other genetic backgrounds may include wild-type genetic backgrounds, genetic backgrounds with deletion mutations, genetic backgrounds with another targeted integration, and genetic backgrounds with non-targeted integrations. Suitable integrations may include without limit nucleic acids encoding drug transporter proteins, Mdr protein, and the like.

### (IV) Applications

A further aspect of the present disclosure encompasses a method for assessing at least one effect of an agent. Suitable agents include without limit pharmaceutically active ingredients, drugs, food additives, pesticides, herbicides, toxins, industrial chemicals, household chemicals, and other environmental chemicals. For example, the effect of an agent may be measured in a “humanized” genetically modified animal, such that the information gained therefrom may be used to predict the effect of the agent in a human. In general, the method comprises contacting a genetically modified animal comprising at least one inactivated chromosomal sequence encoding a neurotransmission-related protein and at least one chromosomally integrated sequence encoding an orthologous neurotransmission-related protein with the agent, and comparing results of a selected parameter to results obtained from contacting a wild-type animal with the same agent. Selected parameters include but are not limited to (a) rate of elimination of the agent or its metabolite(s); (b) circulatory levels of the agent or its metabolite(s); (c)bioavailability of the agent or its metabolite(s); (d) rate of metabolism of the agent or its metabolite(s); (e) rate of clearance of the agent or its metabolite(s); (f) toxicity of the agent or its metabolite(s); (g) efficacy of the agent or its metabolite(s); (h) disposition of the agent or its metabolite(s); and (i) extrahepatic contribution to metabolic rate and clearance of the agent or its metabolite(s).

An additional aspect provides a method for assessing the therapeutic potential of an agent in an animal that may include contacting a genetically modified animal comprising at least one edited chromosomal sequence encoding a neurotransmission-related protein with the agent, and comparing results of a selected parameter to results obtained from a wild-type animal with no contact with the same agent. Selected parameters include but are not limited to a) spontaneous behaviors; b) performance during behavioral testing; c) physiological anomalies; d) abnormalities in tissues or cells; e) biochemical function; and f) molecular structures.

Also provided are methods to assess the effect(s) of an agent in an isolated cell comprising at least one edited chromosomal sequence encoding a neurotransmission-related protein, as well as methods of using lysates of such cells (or cells derived from a genetically modified animal disclosed herein) to assess the effect(s) of an agent. For example, the role of a particular neurotransmission-related protein in the metabolism of a particular agent may be determined using such methods. Similarly, substrate specificity and pharmacokinetic parameter may be readily determined using such methods. Those of skill in the art are familiar with suitable tests and/or procedures.

Yet another aspect encompasses a method for assessing the therapeutic efficacy of a potential gene therapy strategy. That is, a chromosomal sequence encoding a neurotransmission-related protein may be modified such that the potential of a getting neurotransmission disorder is reduced or eliminated. In particular, the method comprises editing a chromosomal sequence encoding a neurotransmission-related protein such that an altered protein product is produced. The genetically modified animal may be exposed to a substance and cellular, and/or molecular responses may be measured and compared to those of a wild-type animal exposed to the same substance. Consequently, the therapeutic potential of the neurotransmission-related gene therapy regime may be assessed.

Still yet another aspect encompasses a method of generating a cell line or cell lysate using a genetically modified animal comprising an edited chromosomal sequence encoding a neurotransmission-related protein. An additional other aspect encompasses a method of producing purified biological components using a genetically modified cell or animal comprising an edited chromosomal sequence encoding a neurotransmission-related protein. Non-limiting examples of biological components include antibodies, cytokines, signal proteins, enzymes, receptor agonists and receptor antagonists

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Genome Editing of 5-HTT in a Model Organism

ZFN-mediated genome editing may be used to study the effects of a “knockout” mutation in a neurotransmission-related chromosomal sequence, such as a chromosomal sequence encoding the 5-HTT protein, in a genetically modified model animal and cells derived from the animal. Such a model animal may be a rat. In general, ZFNs that bind to the rat chromosomal sequence encoding the 5-HTT protein associated with neurotransmission-related disorders may be used to introduce a deletion or insertion such that the coding region of the 5-HTT gene is disrupted such that a functional 5-HTT protein may not be produced.

Suitable fertilized embryos may be microinjected with capped, polyadenylated mRNA encoding the ZFN. The frequency of ZFN-induced double strand chromosomal breaks may be determined using the Cel-1 nuclease assay, as detailed above. The sequence of the edited chromosomal sequence may be analyzed as described above. The development of neurotransmission symptoms and disorders caused by the 5-HTT “knockout” may be assessed in the genetically modified rat or progeny thereof. Furthermore, molecular analyses of neurotransmission-related pathways may be performed in cells derived from the genetically modified animal comprising an ErbB4 “knockout”.

### Example 2

### Generation of a Humanized Rat Expressing a Mutant Form of Human Genes involved in Neurotransmission

Mutations in any of the chromosomal sequences involved in neurotransmission disorders may be used in the generation of a humanized rat expressing a mutant form of the gene. The genes can be 5-HTT, COMT, DRD, SLC6A3, DAO, DTNBP1, GABAa, NMDA, NMDAR, NR1, NR2a, NR2b, mGLUR1, mGLUR2, mGLUR3, mGLUR5, GLUR1, GLUR2, GAD67, GAT1, TCF4, NPAS3, GR1K4, COMT, MAO, DBH, TyrH, CB1, CB2, FAAH, MAGL and combinations thereof. ZFN-mediated genome editing may be used to generate a humanized rat wherein the rat gene is replaced with a mutant form of the human gene comprising the mutation. Such a humanized rat may be used to study the development of the diseases associated with the mutant human protein encoded by the gene of interest. In addition, the humanized rat may be used to assess the efficacy of potential therapeutic agents targeted at the pathway leading to a neurotransmission disorder comprising the gene of interest.

The genetically modified rat may be generated using the methods described in the Examples above. However, to generate the humanized rat, the ZFN mRNA may be co-injected with the human chromosomal sequence encoding the mutant protein into the rat embryo. The rat chromosomal sequence may then be replaced by the mutant human sequence by homologous recombination, and a humanized rat expressing a mutant form of the protein may be produced.

What is claimed is:

1. A genetically modified animal comprising at least one edited chromosomal sequence encoding a neurotransmission-related protein.

2. The genetically modified animal of claim 1, wherein the edited chromosomal sequence is inactivated, modified, or comprises an integrated sequence.

3. The genetically modified animal of claim 1, wherein the edited chromosomal sequence is inactivated such that a functional neurotransmission-related protein is not produced.

4. The genetically modified animal of claim 3, wherein the inactivated chromosomal sequence comprises no exogenously introduced sequence.

5. The genetically modified animal of claim 3, further comprising at least one chromosomally integrated sequence encoding a functional neurotransmission-related protein.

6. The genetically modified animal of claim 1, wherein the neurotransmission-related protein is chosen from 5-HTT, COMT, DRD, SLC6A3, DAO, DTNBP1, GABAa, NMDA, NMDAR, NR1, NR2a, NR2b, mGLUR1, mGLUR2, mGLUR3, mGLUR5, GLUR1, GLUR2, GAD67, GAT1, TCF4, NPAS3, GR1K4, COMT, MAO, DBH, TyrH, CB1, CB2, FAAH, MAGL and combinations thereof.

7. The genetically modified animal of claim 1, further comprising a conditional knock-out system for conditional expression of the neurotransmission-related protein.

8. The genetically modified animal of claim 1, wherein the edited chromosomal sequence comprises an integrated reporter sequence.

9. The genetically modified animal of claim 1, wherein the animal is heterozygous or homozygous for the at least one edited chromosomal sequence.

10. The genetically modified animal of claim 1, wherein the animal is an embryo, a juvenile, or an adult.

11. The genetically modified animal of claim 1, wherein the animal is chosen from bovine, canine, equine, feline, ovine, porcine, non-human primate, and rodent.

12. The genetically modified animal of claim 1, wherein the animal is rat.

13. The genetically modified animal of claim 1, wherein the animal is rat and the protein is a human neurotransmission-related protein.

14. A non-human embryo, the embryo comprising at least one RNA molecule encoding a zinc finger nuclease that recognizes a chromosomal sequence encoding a neurotransmission-related protein, and, optionally, at least one donor polynucleotide comprising a sequence encoding a neurotransmission-related protein.

15. The non-human embryo of claim 14, wherein the neurotransmission-related protein is chosen from 5-HTT, COMT, DRD, SLC6A3, DAO, DTNBP1, GABAa, NMDA, NMDAR, NR1, NR2a, NR2b, mGLUR1, mGLUR2, mGLUR3, mGLUR5, GLUR1, GLUR2, GAD67, GAT1, TCF4, NPAS3, GR1K4, COMT, MAO, DBH, TyrH, CB1, CB2, FAAH, MAGL and combinations thereof; and the embryo is chosen from bovine, canine, equine, feline, ovine, porcine, non-human primate, and rodent.

16. The non-human embryo of claim 14, wherein the embryo is chosen from bovine, canine, equine, feline, ovine, porcine, non-human primate, and rodent.

17. The non-human embryo of claim 14, wherein the embryo is rat and the protein is an ortholog of a human neurotransmission-related protein.

18. A genetically modified cell, the cell comprising at least one edited chromosomal sequence encoding a neurotransmission-related protein.

19. The genetically modified cell of claim 18, wherein the edited chromosomal sequence is inactivated, modified, or comprises an integrated sequence.

20. The genetically modified cell of claim 19, wherein the edited chromosomal sequence is inactivated such that the neurotransmission-related protein is not produced or is not functional.

21. The genetically modified cell of claim 20, further comprising at least one chromosomally integrated sequence encoding a functional neurotransmission-related protein.

22. The genetically modified cell of claim 18, wherein the neurotransmission-related protein is chosen from 5-HTT, COMT, DRD, SLC6A3, DAO, DTNBP1, GABAa, NMDA, NMDAR, NR1, NR2a, NR2b, mGLUR1, mGLUR2, mGLUR3, mGLUR5, GLUR1, GLUR2, GAD67, GAT1, TCF4, NPAS3, GR1K4, COMT, MAO, DBH, TyrH, CB1, CB2, FAAH, MAGL and combinations thereof.

23. The genetically modified cell of claim 18, wherein the cell is heterozygous or homozygous for the at least one edited chromosomal sequence.

24. The genetically modified cell of claim 18, wherein the cell is of bovine, canine, equine, feline, human, ovine, porcine, non-human primate, or rodent origin.

25. The genetically modified cell of claim 18, wherein the cell is of rat origin and the protein is an ortholog of a human neurotransmission-related protein.

26. The genetically modified cell of claim 20, wherein the inactivated chromosomal sequence comprises no exogenously introduced sequence.

27. The genetically modified cell of claim 18, further comprising a conditional knock-out system for conditional expression of the neurotransmission-related protein.

28. The genetically modified cell of claim 18, wherein the edited chromosomal sequence comprises an integrated reporter sequence.

29. A method for assessing the effect of an agent in an animal, the method comprising contacting a genetically modified animal comprising at least one edited chromosomal sequence encoding a neurotransmission-related protein with the agent, and comparing results of a selected parameter to results obtained from contacting a wild-type animal with the same agent, wherein the selected parameter is chosen from:
a) rate of elimination of the agent or its metabolite(s);
b) circulatory levels of the agent or its metabolite(s);
c) bioavailability of the agent or its metabolite(s);
d) rate of metabolism of the agent or its metabolite(s);
e) rate of clearance of the agent or its metabolite(s);
f) toxicity of the agent or its metabolite(s); and
g) efficacy of the agent or its metabolite(s).

30. The method of claim 29, wherein the agent is a pharmaceutically active ingredient, a drug, a toxin, or a chemical.

31. The method of claim 29, wherein the neurotransmission-related protein is chosen from 5-HTT, COMT, DRD, SLC6A3, DAO, DTNBP1, GABAa, NMDA, NMDAR, NR1, NR2a, NR2b, mGLUR1, mGLUR2, mGLUR3, mGLUR5, GLUR1, GLUR2, GAD67, GAT1, TCF4, NPAS3, GR1K4, COMT, MAO, DBH, TyrH, CB1, CB2, FAAH, MAGL and combinations thereof.

32. The method of claim 29, wherein the animal is a rat and the protein is human.

33. A method for assessing the therapeutic potential of an agent in an animal, the method comprising contacting a genetically modified animal comprising at least one edited chromosomal sequence encoding a neurotransmission-related protein with the agent, and comparing results of a selected parameter to results obtained from a wild-type animal with no contact with the same agent, wherein the selected parameter is chosen from:
a) spontaneous behaviors;
b) performance during behavioral testing;
c) physiological anomalies;
d) abnormalities in tissues or cells;
e) biochemical function; and
f) molecular structures.

34. The method of claim 33, wherein the agent is a pharmaceutically active ingredient, a drug, a toxin, a biologically active agent or a chemical.

35. The method of claim 33, wherein the neurotransmission-related protein is chosen from 5-HTT, COMT, DRD, SLC6A3, DAO, DTNBP1, GABAa, NMDA, NMDAR, NR1, NR2a, NR2b, mGLUR1, mGLUR2, mGLUR3, mGLUR5, GLUR1, GLUR2, GAD67, GAT1, TCF4, NPAS3, GR1K4, COMT, MAO, DBH, TyrH, CB1, CB2, FAAH, MAGL and combinations thereof.

36. The method of claim 33, wherein the animal is a rat and the protein is human.
